(19) United States
(12) Patent Application Publication
(10) Pub. No.: US 2009/0326209 A1
(43) Pub. Date: Dec. 31, 2009

(54) Method for isolating and modifying DNA from blood and body fluids

(76) Inventors: Weiwei Li, Lindenhurst, NY (US); Jessica Li, Lindenhurst, NY (US)

Correspondence Address: Weiwei Li, 338 38th Street, Lindenhurst, NY, 11757, US

(21) Appl. No.: 12/287,495

(22) Filed: Oct. 9, 2008

Related U.S. Application Data
(63) Continuation of application No. 11/001,283, filed on Dec. 1, 2004, now abandoned.

(51) Int. Cl. C07H 21/04

(52) U.S. Cl. 536/23.1

(57) ABSTRACT

This invention is related a method for rapidly isolating and modifying DNA from plasma/serum and body fluids. This invention provides a procedure and composition to obtain a high yield of modified DNA for methylation-specific PCR assay by coupling DNA isolation and modification courses.

This is a continuation of Parent application Ser. No. 11/001,283 filed on Dec. 1, 2004 and entitled “Method for isolating and modifying DNA from blood and body fluids”

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

1. Field of the Invention

This invention is related to a method for rapidly isolating and modifying DNA from blood and body fluids. This invention provides a procedure and composition to obtain a high yield of modified DNA for methylation-specific PCR assay by coupling DNA isolation and modification courses.

2. Description of the Related Art

Epigenetic inactivation of the genes plays a critical role in many important human diseases, especially in cancer. The core mechanism for epigenetic inactivation of the genes is methylation of CpG islands in genome DNA. Methylation of CpG islands involves the course in which DNA methyltransferases (Dnmts) transfer a methyl group from S-adenosyl-L-methionine to the fifth carbon position of the cytosines. It was well demonstrated that methylation patterns of DNA from cancer cells are significantly different from those of normal cells. DNA of cancer cells is generally hypomethylated compared to that of normal cells. However DNA of cancer cells could be more methylated than that of normal cells in the selected regions such as in the promoter regions of tumor suppressor genes. Thus, detection of methylation patterns or ratio in the selected genes of cancer cells could lead to discrimination of cancer cells from normal cells, thereby providing an approach to early detection of cancer.

There have been many methods for detection of DNA methylation. The most widely used method among these is methylation-specific PCR (MS-PCR). This assay through chemical modification of DNA, selectively amplifies methylated sequences with primers specific for methylation (Herman et al., Proc. Natl. Acad. Sci. USA 93: 9821-9826, 1996). After PCR a gel-based detection is processed. MS-PCR was recently improved significantly by using the real-time probe system. These improved methods such as MethyLight, Q-MSP (Eads C A et al: Cancer Res, 59: 2302-2306, 1999), and HM-MethyLight (Cottrell SE et al: Nucleic Acids Res. 32: e10, 2004) proved to be more sensitive, specific and quantitative than MS-PCR. However, all existing DNA methylation-based technologies are still not enough to apply to clinical cancer detection, even including MethyLight, a method considered to have potential for clinical application. A critical weakness of these existing methods is that their clinical sensitivity is still too low when a sample from a non-invasive approach is used, such as from plasma/serum or other remote media. It was demonstrated that a cancer at its early stage may release its cells or free DNA into blood through apoptosis, necrosis or local angiogenesis, which establishes a basis for cancer detection using DNA methylation-based technology. The quantity of free circulating DNA from tumor, however, varies from tens of pictogram (pg) to hundreds of nanograms (ng) per ml plasma/serum. Most of them (>90%) range from several hundreds of pg to tens of ng per ml of plasma/serum. The collected DNA will be greatly reduced further by isolation and modification procedures. In general, only 5%-10% of the collected DNA is available as a modified DNA for PCR assay, which may be as low as several pgs (per ml plasma/serum). Based on the limitation level of methylated modified DNA detection (30 pg) in current quantitative MS-PCR technology (i.e.: HM-MethyLight), a complete cancer detection assay (at least 8 target genes) required at least 250 pg of completely methylated modified DNA, that is, 3-5 ng circulating tumor DNA. If considering the condition of that some target genes are only partly methylated (i.e.: 30%), the amount as high as 10-15 ng of circulating tumor DNA is required. It means that 20 ml of plasma/serum or 40 ml of blood must be collected for an assay in order to ensure the correct results available from 90% or greater of the samples. Obviously, low clinical sensitivity of the existing methylation-based cancer detection methods is mainly due to insufficient modified DNA available for PCR assay. To achieve sufficient modified DNA for Q-PCR assay, either a sufficient amount of blood or efficient isolation and modification of DNA must be needed. However, it should be difficult to collect 40 ml of blood for every routine assay for cancer detection. Therefore feasible approach to achieve sufficient modified DNA is only through highly efficient isolation and modification of DNA

Various methods used for DNA isolation from blood or body fluids are available commercially. A standard technique is digestion of the sample with proteinase K followed by phenol/chloroform extraction or more conveniently followed by column purification. Column methods mainly include the High Pure PCR Template Preparation Kit (Roche Diagnostics), QiAamp DNA Mini Kit (Qiagen) and NucleoSpin Blood Kit (Macherey-Nagel Duren). However, DNA recovery by using these methods is about only 40-50% of the original DNA amount because of loss in the handling process. There are also various methods of DNA modification all characterized by bisulfite-treatment. The bisulfite-based DNA modification is used to discriminate between cytosine and methylated cytosine, in which DNA is treated with bisulfite salt to convert cytosine residues to uracil in single-stranded DNA, while methylated cytosine remains same. The bisulfite-based DNA modification basically consists of three processing steps: 1) sulphonation, 2) hydrolytic deamination, and 3) alkali desulphonation. This process involves relatively complex chemistry conditions and it results in serious problems in all of the current bisulfite conversion methods: Time-consuming (usually 16 h) and more critically, severe DNA degradation (84-96%), which results in a low level recovery of modified DNA (Grunau C et al: Nucl Acids Res, 2001). Considering all the problems existing in both currently used DNA isolation and modification method, and furthermore, considering a separated use of existing DNA isolation and modification methods in generating modified DNA, it is impossible to obtain sufficient modified DNA available for a routine methylation-based cancer detection assay. Therefore a more efficient method of DNA isolation and modification is still needed for overcoming problems of existing method to improve methylation-based cancer detection.

### BRIEF SUMMARY OF THE INVENTION

The highly efficient isolation and modification of DNA from blood or body fluids become a critical approach for improving methylation-based cancer detection technology. The present invention provides a method and kit to achieve this approach by a coupled DNA isolation and modification process, comprising the steps of: (1) Isolating genomic DNA, which comprises the interest DNA and background DNA, from plasma, serum, or other body fluids of an individual by using non-chaotropic reagents; (2) Chemically treating DNA in the same tube with a bisulfite salt and the DNA degradation-blocking agents as an essential component, which allows all of unmethylated cytosine bases to be completely converted to uracil in a short time, whereas methylated cytosine bases remain unchanged; (3) Binding chemically modified DNA to a solid phase followed by desulphonation and cleaning; and (4) Eluting the modified DNA with a low salt buffer or water.

Thus the invention allows a highly efficient and fast isolation and modification of genomic DNA from various body fluids, particularly from plasma/serum. This invention is based on the finding that genomic DNA from body fluids can be easily and quickly isolated by using a high concentration of non-chaotropic salt buffer. The invention is also based on the finding that isolated DNA can be directly used for chemical modification with a novel composition provided by this invention. The invention is further based on the finding that a complete modification of genomic DNA can be quickly finished with a high yield of modified DNA by using a novel composition presented in this invention. Therefore the method presented in this invention significantly overcomes the weaknesses existing in the prior technologies and enables a sufficient modified DNA available for a routine cancer detection assay using methylation-based technology.

### BRIEF DESCRIPTION OF THE DRAWINGS

FIG. 1 shows a diagram of the coupled DNA isolation and modification. The process involves the extraction of genomic DNA from plasma/serum and other body fluids and chemical modification of DNA in the same tube. Modified DNA is then captured with a solid carrier surface, purified and eluted.

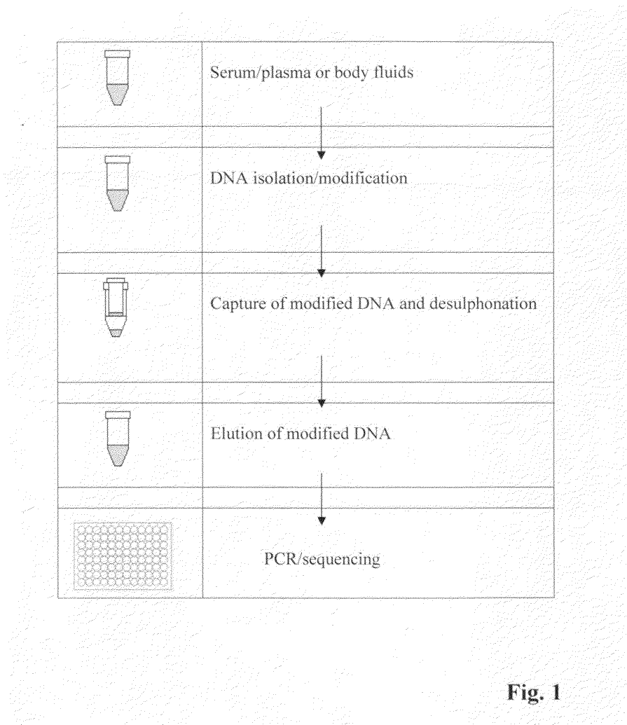

FIG. 2 shows the recovery of DNA from the serum by using the method of this invention. The experiment was carried out as described in Example 1.

FIG. 3 shows an experiment that showed the DNA degradation rate and DNA modification efficiency by using the method of this invention. The experiment was carried out as described in Example 2.

FIG. 4 shows an experiment that showed the required amount of DNA contained in the serum sample for chemical modification using the method of this invention. The experiment was carried out as described in Example 3.

### DETAILED DESCRIPTION OF THE INVENTION

The object of the present invention is to provide a novel method for efficiently isolating and modifying genomic DNA from plasma/serum and other body fluids so that sufficient modified DNA can be available for a routine cancer detection assay using DNA methylation-based technologies. This method is particularly useful for gaining a high yield of modified DNA from a small quantity of starting materials. This method is also particularly useful for fast isolation and modification of DNA in a short time.

In contrast to previous methods for chemically modified DNA preparation (which required a separate process in that genomic DNA is first isolated and purified, and purified DNA is then modified), the method of the present invention, as illustrated in the Example section, generates modified DNA by coupling DNA isolation and modification in a single tube. This coupled process greatly reduces DNA loss, degradation and drastically shortens the process for preparing modified DNA.

The method of the present invention uses the high concentrations of non-chaotropic salts in association with protein enzyme inhibitors to isolate DNA from plasma, serum and other body fluids that include cerebro-spinal fluid, saliva, nasal swab or nasal aspirate, sputum, bronchoalveolar lavage, breast aspirate, cervical swab or vaginal fluid, semen, prostate fluid, and urine. The non-chaotropic salts include, but are not limited to sodium chloride, calcium chloride, lithium chloride, potassium chloride, magnesium chloride, sodium acetate, calcium acetate, lithium acetate, potassium acetate, magnesium acetate, sodium phosphate, calcium phosphate, lithium phosphate, potassium phosphate and magnesium phosphate. A high concentration of non-chaotropic salts is able to cause the dissociation of proteins from DNA and protein molecules precipitation from solution, and further enables DNA to precipitate out of an alcohol solution. It is preferred according to this invention that a sodium salt such as sodium chloride or sodium acetate or their mixture is used in a concentration of 0.1 M to 5 M. It is also preferred according to this invention that the salt solution is an alkalized solution with a pH ranged from 8 to 11. It is more preferred according to this invention that isopropnol or alcohol is used to precipitate DNA.

The DNA isolated in this manner is able to be used for chemical modification after DNA denaturing by heating or alkalized solution such as 0.2 M NaOH. According to this invention the composition of chemical modifying reagents comprise a bisulfite, a potassium salt, or a magnesium salt and an EDTA. The composition of chemical modifying reagents is made in a solution form. According to this invention that bisulfite is selected from sodium bisulfite, potassium bisulfite, ammonium bisulfite, magnesium bisulfite, sodium metabisulfite, potassium metabisulfite, ammonium metabisulfite and magnesium metabisulfite, preferred a sodium metabisulfite from above in the concentrations of 0.5 M to 5 M. According to this invention a potassium salt is selected from potassium chloride, potassium phosphate, and preferred a potassium chloride in the concentrations ranged from 0.1 M to 1 M. According to this invention that an EDTA or an EDTA salt is also selected, preferred EDTA in the concentrations of 1 mM to 100 mM.

An advantage of the composition according to this invention is that unmethylated cytosine residues can be maximally converted to uracil in a single-stranded DNA, while methylated cytosine remains unchanged. Another advantage of the composition according to this invention is that degradation of DNA resulted from chemical, biochemical and thermophilic action in modification is efficiently prevented or reduced. A further advantage of the composition according to this invention is that the DNA modification process is much shorter without interrupting a completed conversion of unmethylated cytosine to uracil and without a significant thermodegradation of DNA resulted from depurination.

In this invention, the temperature for the chemical modification ranges between 50° C. and 80° C., and preferably between 60° C. and 69° C. and more preferably at 65° C. The reaction time set up for chemical modification ranges from 15 min to 4 h, preferably from 30 min to 2 h, more preferably for 1 h.

Once DNA modification is complete, DNA is captured, desulphonated and cleaned. The modified DNA can be captured by a solid matrix selected from silica salt, silica dioxide, silica polymers, glass fiber, celite diatoms and nitrocellulose in the presence of high concentrations of non-chaotropic salts. It is preferred according to this invention that modified DNA is captured with an apparatus comprising a column pre-inserted with a silica gel, or a silica membrane or a silica filter. It is more preferred according to this invention that a silica matrix is pre-treated with alkalized sodium salt solution at a high concentration to enhance the binding of modified DNA. It is further preferred according to this invention that a column is a micro-spin column which fits a 1.5 or 2.0 ml micro-centrifuge tube, and the combination of the column and the micro-centrifuge tube further fits inside a table-top microcentrifuge. After the modified DNA is applied to the column, a binding buffer consisting of non-chaotropic salts at concentrations from 1 M to 5 M can be added to further enhance the binding of the modified DNA to silica matrix. The DNA-bound silica matrix is washed by adding a washing buffer preferably comprising a buffered solution containing 50-90% of ethanol. The modified DNA is further desulphonated on the column with an alkalized solution, preferably sodium hydroxide at concentrations from 10 mM to 300 mM, more preferably at a concentration of 50 mM. Once desulphonation of modified DNA bound to silica matrix has been completed, the column is further washed with the washing buffer 2-3 times. The modified DNA is then eluted from the column and collected into a capped microcentrifuge tube. An elution solution could be DEPC-treated water or TE buffer (10 mM Tris-HCL, pH 8.0 and 1 mM EDTA). Both quality and quantity of eluted modified DNA can be measured by conventional techniques such as pico-green DNA measurement or by PCR amplification.

According to this invention, all of the components for DNA isolation, modification and purification of modified DNA are commercially available. This invention also provides a kit for coupled isolation and modification of DNA from blood and other body fluids, comprising a lysis buffer, a binding buffer and a modification buffer. In one embodiment, the kit further comprises an apparatus with a pre-inserted silica filter to capture modified DNA.

It has been discovered that use of the method of this invention is able to prevent degradation of DNA in DNA modification process, while a complete conversion of cytosine to uracil is performed. It has been also discovered that use of the method of this invention is able to greatly shorten the time required for DNA modification. It has been further discovered that use of the method of this invention can greatly reduce the DNA amount conventionally needed for chemical modification. It has been further discovered that use of the method of this invention can significantly increase yield of modified DNA from the described sample resource.

The method of this invention is applicable for isolating and modifying DNA from whole blood, plasma, serum and buffy coat. The method of this invention is also applicable for isolating and modifying DNA from other body fluids such as cerebro-spinal fluid, saliva, nasal swab or nasal aspirate, sputum, bronchoalveolar lavage, breast aspirate, breast lavage, cervical swab or vaginal fluid, semen, prostate fluid and urine. The method of this invention is further applicable for isolating and modifying DNA from a small amount of cells cultured in a 96-well plate and from media with floating cells or DNA released from apoptotic cells. The plasma or serum can be collected according to the methods described in prior art. The cells contained in other body fluids can be collected by various methods described or by simply centrifugation. By using the method of this invention, the required amount of plasma or serum for an assay point of gene methylation may be as low as 40 ul (assuming the minimum DNA amount in plasma/serum is 0.5 ng/ml). The required number of cells from other body fluids or small in vitro culture may be as few as 5 cells for an assay point of gene methylation.

The method of this invention for isolating and modifying DNA from blood and body fluids is further illustrated in the following examples:

### Example 1

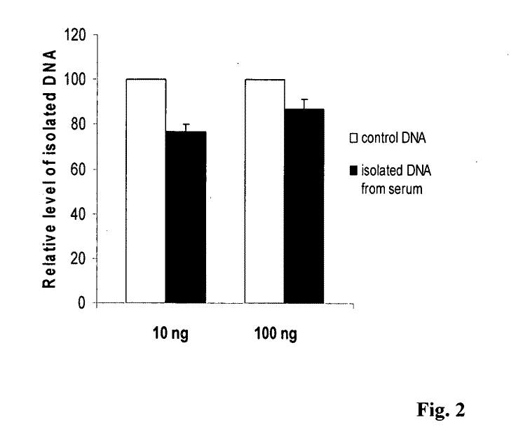

This experiment was carried out in two groups to show the recovery of DNA from serum by using the method of this invention. In group 1, the DNA extracted from blood of a volunteer was added into fetal calf serum (FCS) at different concentrations and mixed. 200 ul of FCS containing different concentrations of DNA were then added to an equal volume of lysis buffer, which comprises a solution of 0.3 M NaOAc and 5 M NaCl with pH 9.0 and 0.25% of proteinase K. The mixture was incubated for 10 min at 65° C. and DNA was then precipitated by adding 0.6 volume of 100% isopropnol followed by centrifugation. Precipitated DNA was kept in the same tube and denatured with 0.2 M NaOH. In comparison, in group 2, the DNA extracted from same blood was directly denatured with 0.2 M of NaOH. Both denatured DNA from group 1 and 2 were then treated with a modification solution for 1 h at 65° C. The modification solution comprises 3.2 M of Na₂S₂O₅, 500 mM of KCl and 0.2 mM EDTA. The solution containing modified DNA was mixed with modified DNA binding buffer comprising non-chaotropic salts and added into a column apparatus with inserted DNA capture filter. Mixed solution passed through the column in a receiver tube by centrifugation. Modified DNA was desulphorated and eluted from the DNA capture filter. The amount of modified DNA from both group 1 and 2 was examined by real-time quantitative PCR. Relative level of isolated DNA from serum is calculated by using the equation: ½^{|ΔCt|}×100%. A pair of primers and a probe designed to amplify both methylated and unmethylated alleles of b-actin were used to quantify DNA. Primer sequences of β-actin are: forward GGAGGTAGGGAGTATATAGGT (SEQ No. 1) and reverse CCAACACACAATAACAAACA (SEQ No. 2). The probe sequence of β-action is: TGATGGAGGAGGTTTAG (SEQ No. 3). As shown in FIG. 2, the level of modified DNA measured in group 1 is approximately 77% and 81% of that in group 2 at 10 and 100 ng of DNA concentration, respectively. Thus an 80% level of DNA recovery from serum can be obtained by using the method of this invention, which is higher than a 50-60% level of DNA recovery from serum by using conventional methods.

### Example 2

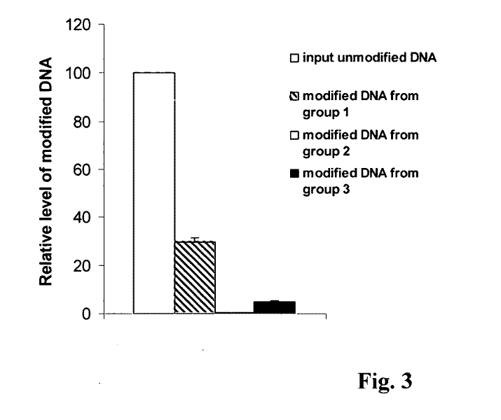

This experiment was carried out in three groups to determine the DNA degradation rate and DNA modification efficiency by using the method of this invention. In group 1, 2 and 3, different concentrations of DNA extracted from blood of a volunteer was denatured. Denatured DNA from group 1 was treated with a modification solution generated in this invention for 1 h at 65° C. The modification solution comprises 3.2 M of Na₂S₂O₅, 500 mM of KCl, and 0.2 mM EDTA. Denatured DNA from group 2 was treated with a conventional modification solution for 1 h at 65° C. Denatured DNA from group 3 was treated with a conventional modification solution for 16 h at 50° C. The conventional modification solution comprises 5 M of sodium bisulfite and 8 mM of hydroquinone. After modification, the solution containing the modified DNA from group 1 were mixed with a modified DNA binding buffer comprising non-chaotropic salts and added into a column apparatus with inserted DNA capture filter. The mixed solution passed through the column by centrifugation in a receiver tube. The modified DNA was desulphorated and eluted from the DNA capture filter. The modified DNA from group 2 and 3 was captured, desulphorated and eluted according to the method described in prior of art (Herman et al., Proc. Natl. Acad. Sci. USA 93: 9821-9826, 1996). The amount of modified DNA from group 1, group 2, and group 3 was examined by real-time quantitative PCR. Relative level of modified DNA of different groups is calculated by using the equation: ½^{ΔCT}×100%. A pair of primers and a probe designed to amplify both methylated and unmethylated β-actin were used to quantify the modified DNA. β-actin primer and probe sequences are described in Example 1. A pair of primers designed to amplify unmodified b-actin was used to quantify unmodified DNA from modification agent-treated DNA and modification agent-untreated input DNA. As shown in FIG. 3, the average amount of modified DNA from group 1 is about 34% of input DNA, while the amount of unmodified DNA is about 0.02% of input DNA. In contrast, the amount of modified DNA from group 2 and group 3 is 97% and 80% less than that obtained from group 1. These results demonstrate that use of the method of this invention can greatly decrease the degradation of DNA in the modification process.

### Example 3

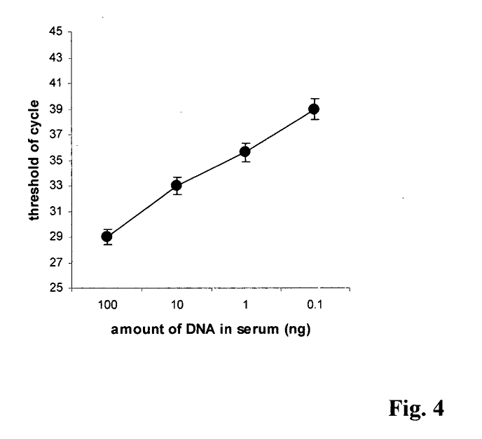

This experiment is carried out to determine the minimum amount of DNA required for chemical modification by using the method of this invention. DNA extracted from blood of a volunteer was added into fetal calf serum (FCS) at concentrations of 0.05, 0.5, 5, and 50 ng/100 ul, respectively. 200 ul of FCS containing different concentrations of DNA were then added to an equal volume of lysis buffer, which comprises a solution of 0.3 M NaOAc and 5 M NaCl with pH 9.0 and 0.25% of proteinase K. The mixture was incubated for 10 min at 65° C. and DNA was then precipitated by adding 0.6 volume of 100% isopropnol followed by centrifugation. Precipitated DNA was kept in same tube and denatured with 0.2 M NaOH. Denatured DNA was then treated with a modification solution for 1 h at 65° C. The modification solution comprises 3.2 M of Na₂S₂O₅, 500 mM of KCl and 0.2 mM EDTA. The solution containing modified DNA was mixed with modified DNA binding buffer comprising non-chaotropic salts and added into a column apparatus with inserted DNA capture filter. Mixed solution passed trough the column in a receiver tube by centrifugation. Modified DNA was desulphorated and eluted from the DNA capture filter with 20 ul water. 2 ul of eluted solution was used for real-time quantitative PCR to examine the amount of modified DNA. A pair of primers and a probe designed to amplify both methylated and unmethylated alleles of β-actin were used to quantify DNA. The sequences of primers and probe are described in Example 1. As shown in FIG. 4, modified DNA was detected even in serum sample containing only 0.1 ng of genomic DNA. Therefore the required amount of DNA contained in a biological sample for chemical modification by using the method of this invention could be less than 0.1 ng.

What is claimed is:

1. A method for modifying DNA isolated from a biological sample of blood and body fluids, comprising: denaturing DNA, treating DNA with a composition comprising an amount of sodium metabisulfite, an amount of a non-chaotropic salt and an amount of an EDTA in an appropriate temperature ranging from 60-69° C. for an appropriate time period from 0.5 to 2 h.

2. According to claim 1, wherein said an amount of sodium metabisulfite is from 0.5 M to 6 M.

3. According to claim 1, wherein said a non-chaotropic salt selected from the group consisting of sodium chloride, lithium chloride, potassium chloride and magnesium chloride.

4. According to claim 1, wherein said a non-chaotropic salt is potassium chloride in an amount of from 0.1 M to 2 M.

5. According to claim 1, wherein said an amount of an EDTA is from 1 mM to 100 mM.

6. According to claim 1, wherein said an appropriate temperature is 65° C.

7. According to claim 1, wherein said an appropriate time period is 1 h.
